(19) United States
(12) Patent Application Publication
(10) Pub. No.: US 2025/0099290 A1
(43) Pub. Date: Mar. 27, 2025

(54) ONE-WAY VALVE FOR URINARY CATHETERS AND THE LIKE

(71) Applicant: Mihna Medical Technologies, Erie, PA (US)

(72) Inventors: Tyler A. Mihna, Erie, PA (US); Corey J. Mihna, Pittsburgh, PA (US)

(73) Assignee: Mihna Medical Technologies, Erie, PA (US)

(21) Appl. No.: 18/475,584

(22) Filed: Sep. 27, 2023

(51) Int. Cl. A61F 5/44

(52) U.S. Cl. CPC A61F 5/4405

(57) ABSTRACT

A one-way flow valve for, e.g., a urinary catheter, has a chamber housing a light bob that is less dense than urine and a heavy bob that is denser than urine and located between the light bob and the chamber's downstream end. When the flow valve is oriented upright with its upstream end higher than its downstream end, the heavy bob abuts the chamber's downstream end in a non-sealing manner that enables urine to flow through the valve in the downstream direction, and, upon urinary backflow, the light bob floats in urine up to form a leakproof seal at an O ring at the chamber's upstream end to prevent backflow of urine through the valve. When the flow valve is oriented upside-down with its downstream end higher than its upstream end, the heavy bob forces the light bob to abut O ring again to form a backflow-preventing leakproof seal.

### BACKGROUND

### Field of the Disclosure

The present disclosure relates to valves and, more specifically but not exclusively, to one-way flow valves for preventing backflow of urine through urinary catheters and the like.

### Description of the Related Art

This section introduces aspects that may help facilitate a better understanding of the disclosure. Accordingly, the statements of this section are to be read in this light and are not to be understood as admissions about what is prior art or what is not prior art.

A urinary catheter is an apparatus used to drain urine by passing a flexible tube through the urethra into the bladder. It is important to prevent backflow of urine from the urine-collection bag through the catheter system back into the patient. As such, many urinary catheters are designed with an anti-reflux chamber which uses a dome design on the top of the catheter drainage bag to help prevent urine reflux from the urine collection bag back into the drainage tube. Unfortunately, conventional urinary catheters have limited functionality especially when the anti-reflux chamber is oriented upside-down, such as when the urine-collection bag is lifted at or above the patient's bladder, in which case, urine may undesirably backflow through the catheter back into the patient.

### SUMMARY

Problems in the prior art are addressed in accordance with the principles of the present disclosure by a one-way flow valve having an interior chamber housing a first bob (e.g., metal ball bearing) that is heavier than the liquid (e.g., urine) and a second bob (e.g., plastic ball bearing) that is lighter than the liquid. When the flow valve is in its desired upright orientation with the valve's upstream end higher than the valve's downstream end, the heavy bob rests against structure at the valve's (lower) downstream end, and the light bob rests on top of the heavy bob, such that urine is able to pass around the heavy and light bobs, and out the downstream end. In the event that the collection bag is lifted at or above the patient's bladder and backflow of urine occurs, the light bob floats on any liquid within the interior chamber up to a resilient sealing element (e.g., a silicone O ring) that forms a leakproof seal that prevents liquid from backflowing out of the valve's (higher) upstream end. When the flow valve is in its upside-down orientation with the downstream end higher than the upstream end, the heavy bob forces the light bob to form the leakproof seal with the sealing element, even when the interior chamber is filled with liquid, to prevent liquid from backflowing out of the valve's (lower) upstream end.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1A is a perspective view of a one-way flow valve for a urinary catheter, according to certain embodiments of the present disclosure;

FIGS. 1B-1D are cross-sectional side views of the flow valve of FIG. 1A;

FIGS. 1E-1G are exploded views of the flow valve of FIG. 1A;

FIG. 1H shows a sliced perspective view of the assembled flow valve of FIG. 1A;

FIG. 2 is a perspective view of the downstream nozzle of the flow valve of FIG. 1A; and

FIG. 3 is a perspective view of one of the ball-bearing guide rails of the flow valve of FIG. 1A.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “contains,” “containing,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

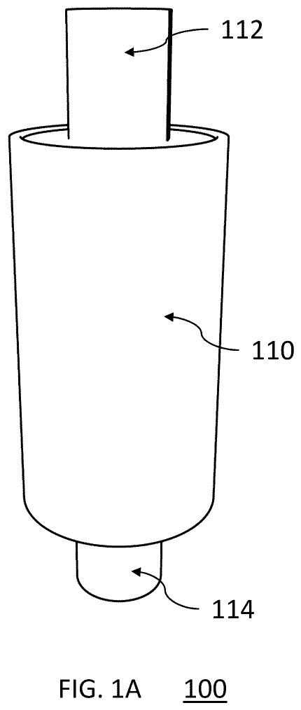

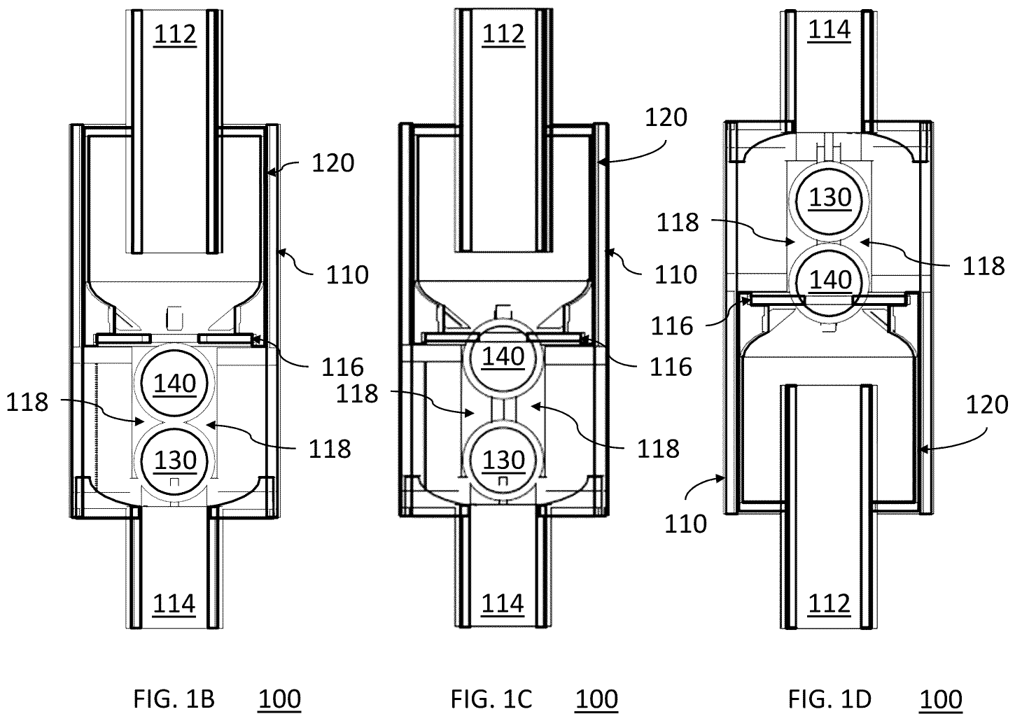

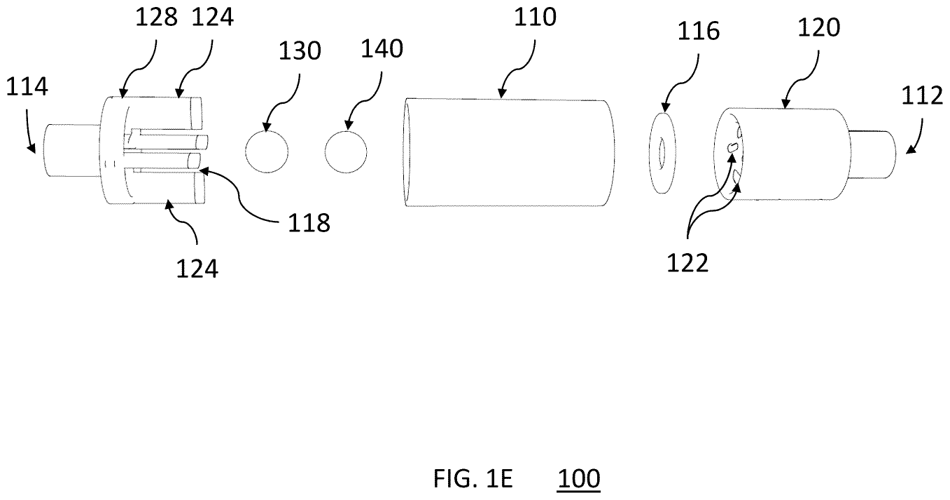

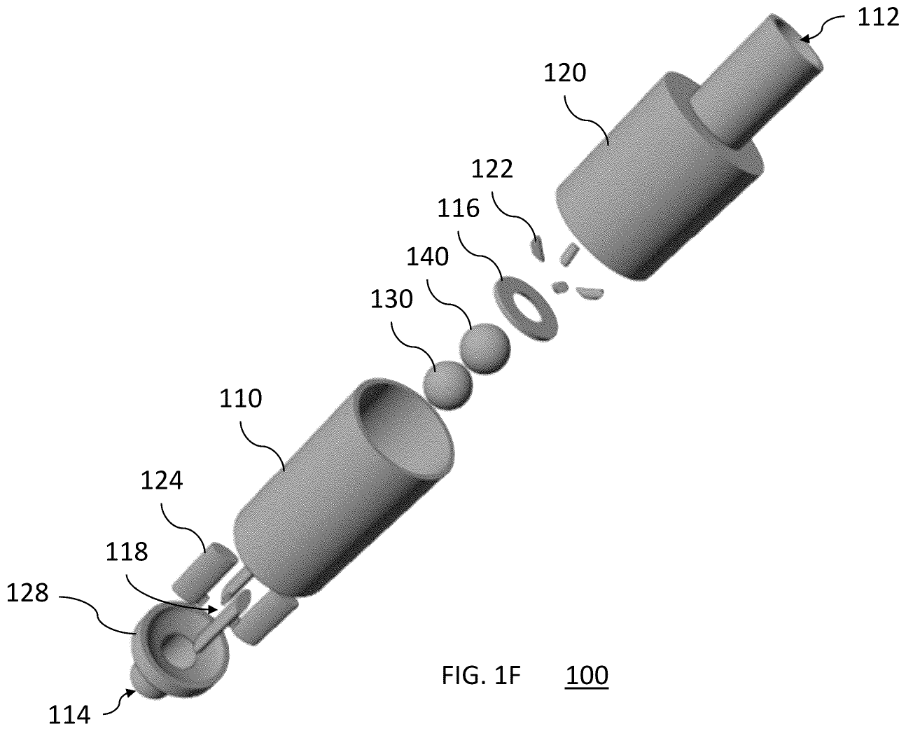

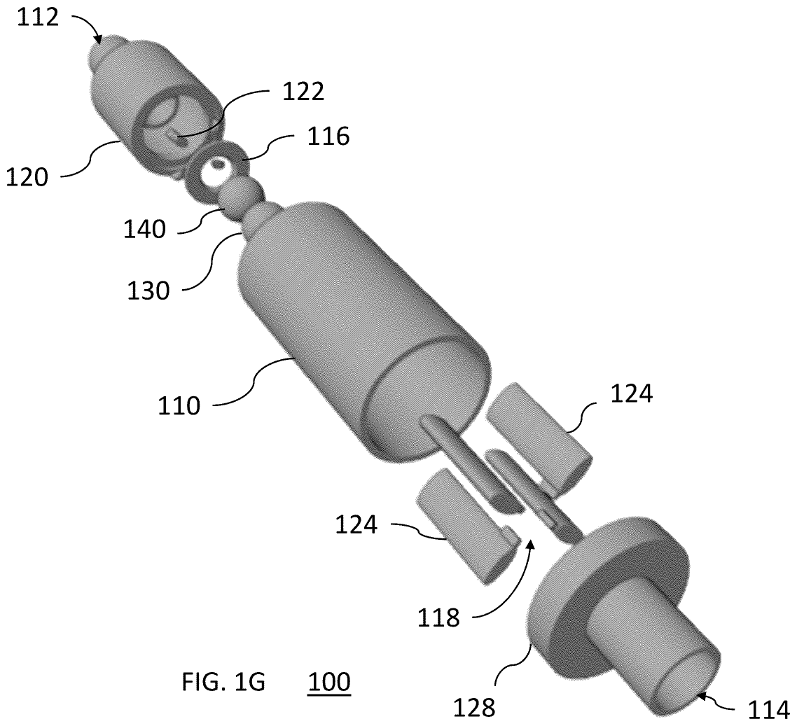

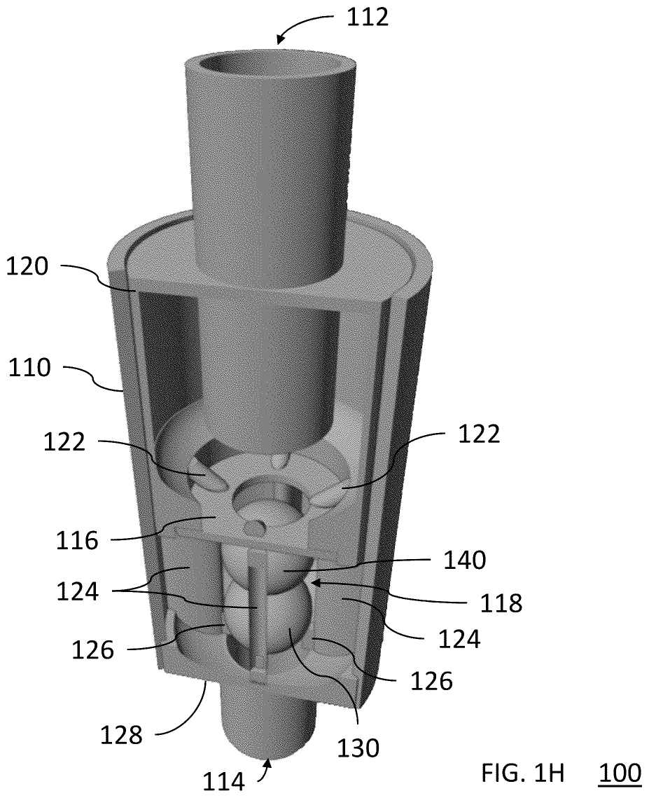

FIG. 1A is a perspective view of a one-way flow valve 100 for a urinary catheter, according to certain embodiments of the present disclosure. As shown in FIG. 1A, the flow valve 100 has a body component 110 between (i) an upstream end 112 (i.e., the end that receives urine from a catheterized patient through upstream catheter tubing (not shown) connected to the upstream end 112) and (ii) a downstream end 114 (i.e., the end that dispenses urine from the flow valve 100 into a urine-collection system (not shown) through downstream catheter tubing (not shown) connected to the downstream end 114).

FIGS. 1B-1D are cross-sectional side views of the flow valve 100 of FIG. 1A. FIGS. 1B and 1C show the flow valve 100 in its preferred, upright (aka “0-degree”) orientation with the upstream end 112 higher than the downstream end 114, while FIG. 1D shows the flow valve 100 in its upside-down (aka “180-degree”) orientation with the downstream end 114 higher than the upstream end 112.

As shown in FIGS. 1B-1D, between the upstream and downstream ends 112 and 114 is a hollow chamber 118 having a resilient sealing element 116 (e.g., a silicone O ring) mounted in a recessed lip on upstream nozzle 120. Also located within the chamber 118 are (i) an untethered, so-called “heavy” bob 130 (e.g., a stainless steel ball bearing) whose density is greater than the density of urine and (ii) an untethered, so-called “light” bob 140 (e.g., a hollow polypropylene ball bearing) whose density is lesser than the density of urine, where the heavy bob 130 is located between the light bob 140 and the downstream end 114 of the flow valve 100. As described further below, the valve 100 is designed to enable urine to flow out of the patient around both the light bob 140 and the heavy bob 130 within the chamber 118.

FIG. 1B represents a situation in which (i) the flow valve 100 is in its upright orientation and (ii) the chamber 118 is empty. In this situation, due to gravity, the heavy bob 130 abuts structure at the downstream end of the chamber 118 with the light bob 140 resting on the heavy bob 130. As described further below, the structure at the downstream end of the chamber 118 is designed to enable urine to flow out of the valve's downstream end 114 even when the heavy bob 130 abuts that structure. In the situation of FIG. 1B, urine flow from the patient that enters the valve 100 at the upstream end 112 will flow, due to gravity, around the light bob 140 and the heavy bob 130 and out the downstream end 114.

FIG. 1C represents a situation in which (i) the flow valve 100 is in its upright orientation and (ii) the chamber 118 is filled with enough urine from backflow such that the light bob 140 floats in the urine to abut the sealing element 116 at the upstream end of the chamber 118. Note that the heavy bob 130 remains abutting the structure at the downstream end of the chamber 118 due to its density being greater than that of urine. In this situation, with the light bob 140 abutting the sealing element 116, urinary backflow applied by the urine onto the light bob 140 will result in the light bob 140 forming a leakproof seal at the interface between the light bob 140 and the sealing element 116 that prevents or at least significantly limits urine from backflowing through the valve 100 back into the patient.

As represented in FIG. 1D, with the flow valve 100 in its upside-down orientation, due to gravity, the heavy bob 130 forces the light bob 140 to abut the resilient sealing element 116 with sufficient force to again form the leakproof seal at the interface between the sealing element 116 and the light bob 140, thereby preventing or at least significantly limiting backflow of urine through the flow valve 100 back into the patient. Note that the heavy bob 130 is sufficiently heavy to generate a leakproof seal even when the chamber 118 is filled with urine.

FIGS. 1E-1G are exploded views of the flow valve 100 of FIG. 1A. FIG. 1F is an exploded, perspective view of the flow valve 100 looking from the upstream end 112 towards the downstream end 114, while FIG. 1G is an exploded, perspective view of the flow valve 100 looking from the downstream end 114 towards the upstream end 112. As shown in FIGS. 1E-1G, flow value 100 comprises:
Upstream end 112, which attaches to upper catheter system (not shown);
Upstream nozzle 120, which attaches to upstream end 112;
Four rigid, anti-seize bearing pegs 122, which are mounted within the upstream nozzle 120;
Silicone O-ring 116, which functions as the resilient sealing element;
Hollow polypropylene ball bearing 140, which functions as the light bob;
Stainless steel ball bearing 130, which functions as the heavy bob;
Body component 110;
Four ball-bearing guide rails 124, which are mounted at the downstream end of the body component 110 and keep the ball bearings 130 and 140 centered as they move up and down within the chamber 118; and
Downstream nozzle 128 which attaches to downstream end 114; and
Downstream end 114, which attaches to lower catheter system; (not shown).

FIG. 1H shows a sliced perspective view of the assembled flow valve 100 of FIG. 1A looking from the upstream end 112 towards the downstream end 114. As shown in FIG. 1H:
The upstream nozzle 120 fits within the body component 110;
The O ring 116 is glued onto a circular lip at the downstream end of the upstream nozzle 120 using a suitable silicone adhesive and positioned above the seal in its upright position;
The anti-seize bearing pegs 122, which prevent the light ball bearing 140 from moving further upstream, are mounted at the downstream end of the upstream nozzle 120; and
Each ball-bearing guide rail 124, which is mounted within the downstream portion of the body component 110, has a rigid stop 126 that stops the downstream travel of the heavy ball bearing 130.

In some implementations, the body component 110, the four guide rails 124, and the downstream nozzle 128 are formed as a single unitary structure.

As represented in FIG. 1H, the chamber 118 is the volume within the body component 110 between (i) the O ring 116 at the downstream end of the upstream nozzle 120 and (ii) the upstream end of the downstream nozzle 128, with the guide rails 124 keeping the two ball bearings 130 and 140 centered within the chamber 118. The longitudinal dimension (i.e., height) of the chamber 118 is slightly larger than the sum of the diameters of the two ball bearings 130 and 140, such that, with the flow valve 100 in its upright orientation and the heavy ball bearing 130 resting against the four stops 126 at the downstream end of the chamber 118, the light ball bearing 140 is able to float on urinary backflow within the chamber 118 the small distance up to the O ring 116.

With the outer diameter of the chamber 118 being larger than the diameters of the two ball bearings 130 and 140 and with the stops 126 at the downstream ends of the guide rails 124, urine is able to flow around the two ball bearings 130 and 140 through chamber 118 and out the valve's downstream end 114 when the flow valve's upstream end 112 is higher than its downstream end 114, i.e., with the orientation within the range of 0 to 89 degrees.

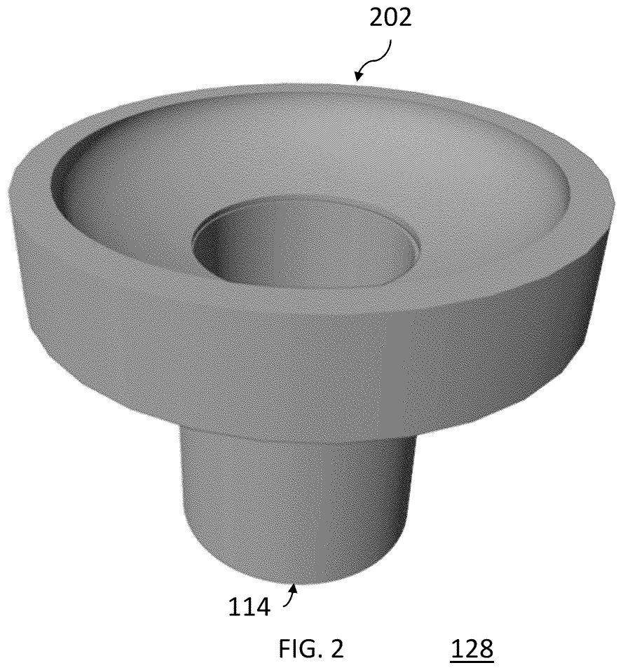

FIG. 2 is a perspective view of the downstream nozzle 128 of the flow valve 100 of FIG. 1A. As shown in FIG. 2, the downstream nozzle 128 has a concave shape at its upstream end 202 that helps to funnel urine out the valve's downstream end 114.

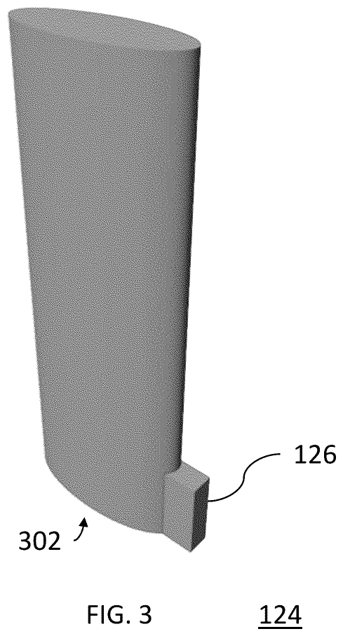

FIG. 3 is a perspective view of one of the ball-bearing guide rails 124 of the flow valve 100 of FIG. 1A. As shown in FIG. 3, each guide rail 124 has a stop 126 as its downstream end 302.

One possible implementation of the flow valve 100 of FIG. 1A has the following dimensions:
Diameter of ball bearings 130 and 140: 10 mm
Height of chamber 118: 20 mm
Diameter of chamber 118: 26 mm
Distance from one guide rail 124 to its opposing guide rail 124: 10.1 mm.
Width of guide rails 124: 3 mm at widest point; 6.5 mm from where guide rail 124 touches body component 110 to end of guide rail 124
Size of stops 126: 3.5 mm height, 1 mm width
Outer diameter of O ring: 20 mm
Inner diameter of O ring: 8 mm
Thickness of O ring: 1/32″
   The heavy ball bearing 130 may be a Stainless Steel Precision Ball from Bal-tec of Canonsburg, Pennsylvania; the light ball bearing 140 may be a Hollow Polypropylene Plastic Precision Ball from Precision Plastic Ball Company of Franklin Park, Illinois; and the O ring 116 may be punched to proper dimension from a silicone sheet from Zoro Corporation of Buffalo Grove, Illinois.

Although the disclosure has been described in the context of situations in which the flow valve 100 is oriented in either its upright, 0-degree orientation or its upside-down, 180-degree orientation, the flow value 100 will also operate appropriately for orientations between 0 and 180 degrees. For example, for orientations between 0 and 90 degrees, the heavy ball bearing 130 will still rest on the stops 126 at the downstream ends of the guide rails 124, and the light ball bearing 140 will still float on any urine within the chamber 118 up to the O ring 116. In addition, for orientations between 90 and 180 degrees, the heavy ball bearing 130 will still force the light ball bearing 140 to abut the O ring 116, albeit with diminishing pressure as the orientation approaches 90 degrees. In orientations near or even at 90 degrees (i.e., horizontal), any significant backpressure of urine within the chamber 118 will force the light ball bearing 140 to form the leakproof seal with the O ring 116.

Although the present disclosure has been described in the context of a one-way flow valve in which the heavy bob 130 is a stainless steel ball bearing and the light bob 140 is a hollow polypropylene ball bearing, those skilled in the art will understand that the disclosure can be implemented with alternative designs. For example, instead of a stainless steel ball bearing, the heavy bob may be made of other materials that are denser than the liquid (e.g., urine) and/or with shapes other than spheres, such as (without limitation) cylinders. Similarly, instead of a hollow polypropylene ball bearing, the light bob 140 can be hollow or solid, made of other materials that are less dense than the liquid (e.g., urine), and/or with shapes other than spheres, such (without limitation) cylinders.

Although the disclosure has been described in the context of the flow valve of FIG. 1A, the disclosure is not limited to that particular design. For example and without limitation:
The anti-seize pegs 122 that hold the O ring 116 in place may be replaced by any other suitable structures that provide that function;
The guide rails 124 that keep the ball bearings 130 and 140 centered within the chamber 118 may be replaced by any other suitable structures that provide that function;
The O ring 116 that forms a leakproof seal with the light ball bearing 140 may be replaced by any other suitable structure that provides that function;
The guide rail stops 126 that stop the downstream movement of the heavy ball bearing 130 may be replaced by any other suitable structure that provides that function;
The heavy ball bearing 130 that sinks in urine and causes the light ball bearing 140 to form the leakproof seal with the O ring 116 when the flow valve 100 is in its upside-down orientation may be replaced by any other suitable structure that provides that function (including non-spherical structures); and
The light ball bearing 140 that floats in urine and forms the leakproof seal with the O ring 116 may be replaced by any other suitable structure that provides that function (including non-spherical structures).

Although the present disclosure has been described in the context of one-way flow valves for urinary catheters, the present disclosure may be implemented in other contexts in which one-way flow is desired with backflow being prevented or at least inhibited for liquids other than urine.

In certain embodiments of the present disclosure, an apparatus comprises a one-way flow valve (e.g., 100) for a liquid having a liquid density, the flow valve comprising (i) a chamber (e.g., 118) having an upstream end (e.g., 112) for receiving the liquid and a downstream end (e.g., 114) for dispensing the liquid; (ii) a heavy bob (e.g., 130) located within the chamber and having a density greater than the liquid density; and (iii) a light bob (e.g., 140) located within the chamber and having a density lesser than the liquid density, wherein the heavy bob is located between the light bob and the downstream end of the chamber. When the flow valve is oriented with the upstream end higher than the downstream end, (a) the heavy bob will abut the downstream end of the chamber in a non-sealing manner, and the liquid will flow through the chamber and out of the downstream end, and (b) when there is sufficient liquid backpressure, the light bob will float in the liquid up to abut the upstream end of the chamber in a sealing manner that inhibits the liquid from backflowing out of the chamber at the upstream end. When the flow valve is oriented with the downstream end higher than the upstream end, the heavy bob forces the light bob to abut the upstream end of the chamber in the sealing manner that inhibits the liquid from backflowing out of chamber at the upstream end.

In at least some of the above embodiments, the heavy and light bobs have spherical shapes.

In at least some of the above embodiments, the heavy bob is a stainless steel ball bearing; and the light bob is a hollow polypropylene ball bearing.

In at least some of the above embodiments, the diameters of the heavy and light bobs are smaller than the inner diameter of the chamber; the chamber has interior guide rails (e.g., 124) that guide the heavy and light bobs along a longitudinal direction within the chamber; and the chamber and the guiderails are designed to enable the liquid to flow within the chamber around the heavy and light bobs.

In at least some of the above embodiments, the downstream end of the chamber has rigid stops (e.g., 126) that receive the heavy bob in the non-sealing manner; and the upstream end of the chamber has a resilient sealing element (e.g., 116) that receives the light bob in the sealing manner.

In at least some of the above embodiments, the upstream end has structure (e.g., 122) that limits the deformation of the resilient sealing member when the light bob is forced into the resilient sealing element (e.g., 116) by either (i) liquid backflow or (ii) the heavy bob forcing the light bob onto the resilient sealing element.

In at least some of the above embodiments, the rigid stops are part of interior guide rails (e.g., 124) that guide the heavy and light bobs within the chamber; and the sealing element is a silicone O ring.

In at least some of the above embodiments, the apparatus connects to a urinary catheter.

Unless explicitly stated otherwise, each numerical value and range should be interpreted as being approximate as if the word “about” or “approximately” preceded the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence. Likewise, additional steps may be included in such methods, and certain steps may be omitted or combined, in methods consistent with various embodiments of the disclosure.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements. For example, the phrases “at least one of A and B” and “at least one of A or B” are both to be interpreted to have the same meaning, encompassing the following three possibilities: 1—only A; 2—only B; 3—both A and B.

All documents mentioned herein are hereby incorporated by reference in their entirety or alternatively to provide the disclosure for which they were specifically relied upon.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

As used herein and in the claims, the term “provide” with respect to an apparatus or with respect to a system, device, or component encompasses designing or fabricating the apparatus, system, device, or component; causing the apparatus, system, device, or component to be designed or fabricated; and/or obtaining the apparatus, system, device, or component by purchase, lease, rental, or other contractual arrangement.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the technology of the disclosure. It is intended that the following claims define the scope of the invention and that methods and structures within the scope of these claims and their equivalents be covered thereby.

What is claimed is:

1. An apparatus comprising a one-way flow valve (e.g., 100) for a liquid having a liquid density, the flow valve comprising:
a chamber (e.g., 118) having an upstream end (e.g., 112) for receiving the liquid and a downstream end (e.g., 114) for dispensing the liquid;
a heavy bob (e.g., 130) located within the chamber and having a density greater than the liquid density; and
a light bob (e.g., 140) located within the chamber and having a density smaller than the liquid density, wherein:
the heavy bob is located between the light bob and the downstream end of the chamber;
when the flow valve is oriented with the upstream end higher than the downstream end:
the heavy bob will abut the downstream end of the chamber in a non-sealing manner, and the liquid will flow into the upstream end, through the chamber, and out of the downstream end; and
when there is sufficient liquid backpressure, the light bob will float in the liquid up to abut the upstream end of the chamber in a sealing manner that inhibits the liquid from backflowing out of the chamber at the upstream end; and
when the flow valve is oriented with the downstream end higher than the upstream end, the heavy bob forces the light bob to abut the upstream end of the chamber in the sealing manner that inhibits the liquid from backflowing out of chamber at the upstream end.

2. The apparatus of claim 1, wherein the heavy and light bobs have spherical shapes.

3. The apparatus of claim 2, wherein:
the heavy bob is a stainless steel ball bearing; and
the light bob is a hollow polypropylene ball bearing.

4. The apparatus of claim 1, wherein:
the diameters of the heavy and light bobs are smaller than the inner diameter of the chamber;
the chamber has interior guide rails (e.g., 124) that guide the heavy and light bobs along a longitudinal direction within the chamber; and
the chamber and the guiderails are designed to enable the liquid to flow within the chamber around the heavy and light bobs.

5. The apparatus of claim 1, wherein:
the downstream end of the chamber has stops (e.g., 126) that receive the heavy bob in the non-sealing manner; and
the upstream end of the chamber has a sealing element (e.g., 116) that receives the light bob in the sealing manner.

6. The apparatus of claim 5, wherein the upstream end has structure (e.g., 122) that limits the deformation of the sealing member when the light bob is forced into the sealing element by either (i) liquid backflow or (ii) the heavy bob forcing the light bob onto the sealing element.

7. The apparatus of claim 5, wherein:
the stops are part of interior guide rails (e.g., 124) that guide the heavy and light bobs within the chamber; and
the sealing element is a silicone O ring.

8. The apparatus of claim 1, wherein the apparatus is any device in which liquid is transferred.

9. The apparatus of claim 1, wherein the apparatus is a urinary catheter.

10. The apparatus of claim 1, wherein:
the heavy bob is a stainless steel ball bearing;
the light bob is a hollow polypropylene ball bearing;
the diameters of the heavy and light bobs are smaller than the inner diameter of the chamber;
the chamber has interior guide rails (e.g., 124) that guide the heavy and light bobs along a longitudinal direction within the chamber;
the chamber and the guiderails are designed to enable the liquid to flow within the chamber around the heavy and light bobs;
the downstream end of the chamber has rigid stops (e.g., 126) that receive the heavy bob in the non-sealing manner;
the upstream end of the chamber has a resilient sealing element (e.g., 116) that receives the light bob in the sealing manner;
the upstream end has structure (e.g., 122) that limits the deformation of the resilient sealing member when the light bob is forced into the sealing element by either (i) liquid backflow or (ii) the heavy bob forcing the light bob onto the sealing element;
the rigid stops are part of interior guide rails (e.g., 124) that guide the heavy and light bobs within the chamber;
the sealing element is a silicone O ring; and
the apparatus is a urinary catheter.
